(19) United States
(12) Patent Application Publication
(10) Pub. No.: US 2021/0322144 A1
(43) Pub. Date: Oct. 21, 2021

(54) Side-Attachment Cradle for Veterinary Surgical Tables

(71) Applicant: Rosemary Koop, Oregon City, OR (US)

(72) Inventor: Rosemary Koop, Oregon City, OR (US)

(21) Appl. No.: 16/315,771

(22) PCT Filed: Sep. 21, 2017
(86) PCT No.: PCT/IB2017/055746

§ 371 Date: Jan. 7, 2019

(51) Int. Cl. A61D 3/00

(52) U.S. Cl. CPC A61D 3/00

(57) ABSTRACT

A side-attachment cradle for veterinary surgical tables includes a platform, a first length-adjustable skirt, a second length-adjustable skirt, at least one first skirt-positioning mechanism, at least one second skirt-positioning mechanism, and a surgical-table adapter. The platform, the first length-adjustable skirt, and the second length-adjustable skirt enclose a holding area for securely holding an animal during necessary surgical procedures. The first skirt-positioning mechanism positions the first length-adjustable skirt by adjusting a first angle and a first offset-distance from the platform's central axis. The second skirt-positioning mechanism positions the second length-adjustable skirt by adjusting a second angle and a second offset distance from the platform's central axis, thereby changing the effective volume enclosed by the holding area. The surgical-table adapter allows the present invention to selectively fasten onto a conventional veterinary surgical table. More specifically, the surgical-table adapter perimetrically engages onto the edges of the veterinary surgical table.

The current application is a 371 of international Patent Cooperation Treaty (PCT) application PCT/IB2017/055746 filed on Sep. 21, 2017. The international Patent Cooperation Treaty (PCT) application PCT/IB2017/055746 claims a priority to the U.S. Provisional Patent application Ser. No. 62/297,634 filed on Sep. 21, 2016.

### FIELD OF THE INVENTION

The present invention generally relates to side-attachment cradle for a veterinary surgical table. In particular, a side-attachment cradle enables a volume-adjustable holding area for restraining animals during necessary surgical procedures.

### BACKGROUND OF THE INVENTION

In the field of animal welfare, it is often the case that when an animal is injured, sick, or in general discomfort, the animal can get agitated. An injured animal is aggravated very easily and oftentimes become very aggressive towards anyone who tries to get near it, such as caretakers or medical personnel. Further, the animal can also start struggling and moving around in a violent manner to try to get away from its caretaker or owner. This can result in the animal exacerbating its injuries and putting itself in a life-threatening situation.

Even after the animal is transported to a veterinary facility, several medical workers are required to physically restrain the animal before necessary surgical procedures can be performed. This is an especially challenging task since most animals are instinctively wary of veterinarians. In many cases, regular harnesses and restrains cannot be adapted to secure animals of varying sizes. Additionally, a conventional surgical table with a flat table top often lacks the means to properly position the animal. Even after the animal has been initially subdued or sedated, medical workers must physically hold the animal in a stabilized position. If the workers fail to keep the animal in a stable position, the surgeon may accidentally damage the animal's vital organs. Thus, a system for securely retaining an animal in a stabilized position during necessary surgical procedures is needed.

The present invention is a side-attachment cradle for a veterinary surgical table that selectively engages with a conventional veterinary surgical table for securely holding large animals such as a large breed of dog for necessary surgical procedures. The present invention restrains and positions the animal in the proper orientation needed to perform necessary surgical procedures. The present invention encloses a volume-adjustable holding area that conforms to the specific body dimensions of the animal being restrained. As such, the present invention physically supports the animal and positions it in a stabilized position, in convenient reach of a surgeon. The present invention can also be easily retrofitted into an existing veterinary surgical table or may be available as a standalone unit. Further, the present invention is composed of sterile and surgical grade materials that can be easily disinfected.

### BRIEF DESCRIPTION OF THE DRAWINGS

FIG. 1 is a front perspective view of the manually-actuated embodiment of the present invention.

FIG. 2 is a front planform view of the manually-actuated embodiment of the present invention showing the first length-adjustable skirt and the second length-adjustable skirt being raised to enclose a low volume holding area.

FIG. 3 is a front planform view of the manually-actuated embodiment of the present invention showing the first length-adjustable skirt and the second length-adjustable skirt being lowered to enclose a high volume holding area.

FIG. 4 is a side planform view of the manually-actuated embodiment of the present invention showing the skirt extension, skirt base, front pawl arm, and the rear pawl arm configure to reduce the length of the first length-adjustable skirt.

FIG. 5 is a side planform view of the manually-actuated embodiment of the present invention showing the skirt extension, skirt base, front pawl arm, and the rear pawl arm configure to increase the length of the first length-adjustable skirt.

FIG. 6 is a front perspective view of the automatically-actuated embodiment of the present invention showing the position of the first skirt-positioning mechanism and the second skirt positioning mechanism.

FIG. 7 is a front planform view of the automatically-actuated embodiment of the present invention showing the first length-adjustable skirt and the second length-adjustable skirt being raised to enclose a low volume holding area.

FIG. 8 is a front planform view of the automatically-actuated embodiment of the present invention showing the first length-adjustable skirt and the second length-adjustable skirt being lowered to enclose a high volume holding area.

FIG. 9 is a side planform view of the automatically-actuated embodiment of the present invention showing the motorized lifting mechanism lifting configured to reduce the length of the first length-adjustable skirt.

FIG. 10 is a side planform view of the automatically-actuated embodiment of the present invention showing the motorized lifting mechanism lifting configured to increase the length of the first length-adjustable skirt.

### DETAILED DESCRIPTION OF THE INVENTION

All illustrations of the drawings are for the purpose of describing selected versions of the present invention and are not intended to limit the scope of the present invention.

The present invention is a side-attachment cradle for a veterinary surgical table. The present invention is configured to slidably engage a conventional veterinary surgical table. The present invention cradles/holds a large animal or a large breed of dog in a stabilized position during necessary surgical procedure, thereby reducing or eliminating the need for veterinary workers to manually restrain the animal.

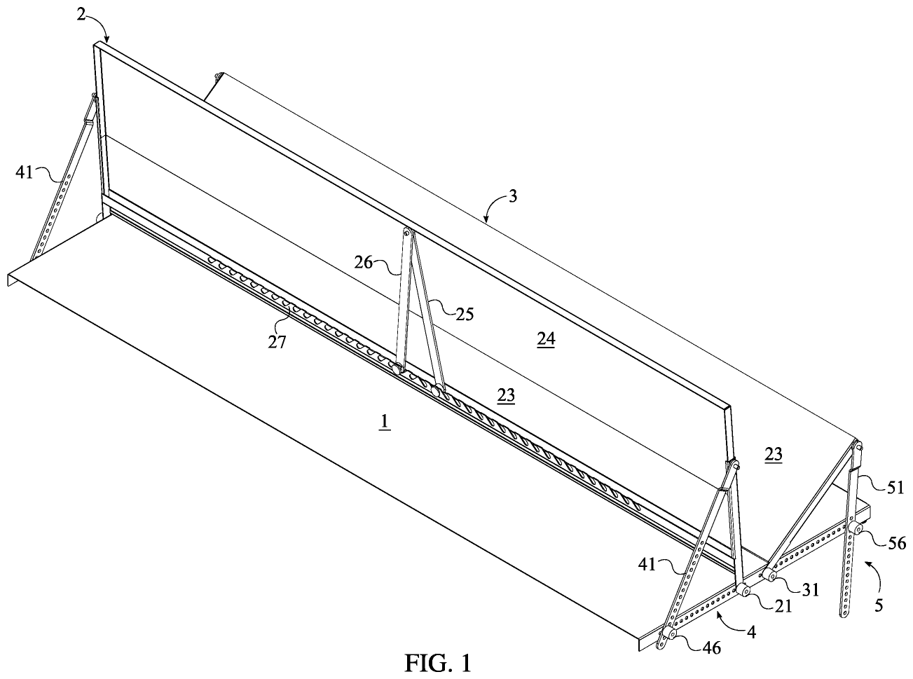

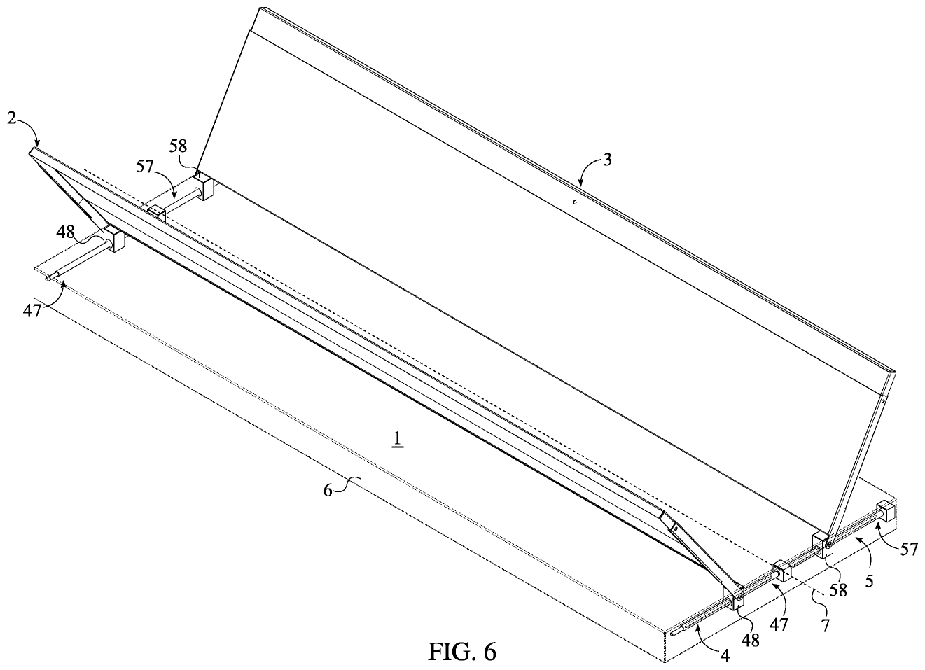

In reference to FIG. 1 and FIG. 6, the preferred embodiment of the present invention comprises a platform 1, a first length-adjustable skirt 2, a second length-adjustable skirt 3, at least one first skirt-positioning mechanism 4, at least one second skirt-positioning mechanism 5, and a surgical-table adapter 6. The platform 1, the first length-adjustable skirt 2, and a second length-adjustable skirt 3 enclose a holding area which securely retains the animal. The first length-adjustable skirt 2 and the second length-adjustable skirt 3 are positioned adjacent to the platform 1. The holding area is located between the first length-adjustable skirt 2 and the second length-adjustable skirt 3 and is longitudinally aligned to the platform 1. As a result, the holding area can be used to restrain an animal in a supine or prone position on top of the platform 1. In both the supine and prone position, the first length-adjustable skirt 2 and the second length-adjustable skirt 3 laterally support the torso of the animal. However, in a supine position, the platform 1 supports the abdomen of the animal. In a prone position, the platform 1 supports the back of the animal. The first length-adjustable skirt 2 and the second length-adjustable skirt 3 may be composed of sterile surgical grade materials that can be easily disinfected.

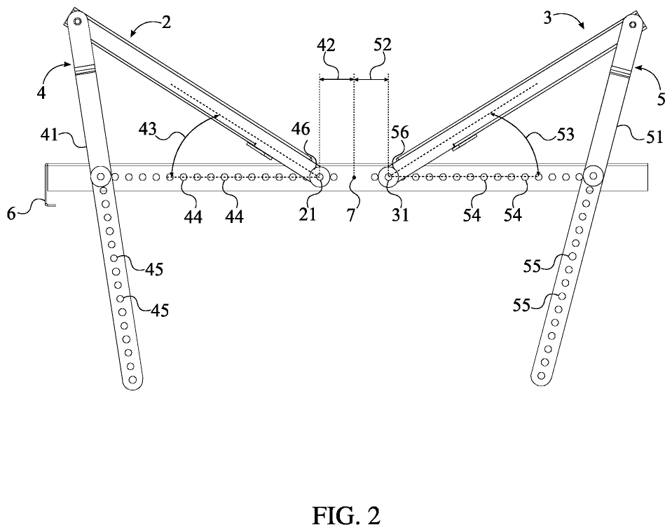

FIG. 1-2 shows the first skirt-positioning mechanism 4 operatively integrated between the first length-adjustable skirt 2 and the platform 1. The first skirt-positioning mechanism 4 may be an electromechanical, hydraulic, or manual actuation mechanism for adjusting the position of the first length-adjustable skirt 2. More specifically, the first skirt-positioning mechanism 4 adjusts a first angle 43 between the first length-adjustable skirt 2 and the platform 1 and a first offset distance 42 between the first rotation axis 21 and a central axis 7 of the platform 1. Further, the second skirt-positioning mechanism 5 is operatively integrated between the second length-adjustable skirt 3 and the platform 1. Like the first skirt-positioning mechanism 4, the second skirt-positioning mechanism 5 may be an electromechanical, hydraulic, or manual actuation mechanism for adjusting the position of the second length-adjustable skirt 3. More specifically, the second skirt-positioning mechanism 5 adjusts a second angle 53 between the second length-adjustable skirt 3 and the platform 1 and a second offset distance 52 between the second rotation axis 31 and the central axis 7 of the platform 1.

The surgical-table adapter 6 is mounted adjacent to the platform 1, opposite the first length-adjustable skirt 2 and the second length-adjustable skirt 3. The surgical-table adapter 6 preferably utilizes a snap locking mechanism that can perimetrically engage the edges of a veterinary surgical table. However, any selectively fastening mechanism that securely fastens the platform 1 to a veterinary surgical table may be utilized. Alternately, the platform 1 may be modified to allow the present invention to stand on its own without being attached to veterinary surgical table. This may be achieved through the use of legs mounted on the corners of the platform 1.

In the preferred embodiment of the present invention, the at least one first skirt-positioning mechanism 4 is a pair of first skirt-positioning mechanisms 4. More specifically, the pair of first skirt-positioning mechanisms 4 is positioned opposite each other across the platform 1. This allows the pair of first skirt-positioning mechanisms 4 to support the longitudinal ends of the first length-adjustable skirt 2.

Similarly, the preferred embodiment of the at least one second skirt-positioning mechanism 5 is a pair of second skirt-positioning mechanisms 5. FIG. 1 shows the pair of second skirt-positioning mechanisms 5 positioned opposite each other across the platform 1. This allows the pair of second skirt-positioning mechanisms 5 to similarly support the terminal portions of the of the second length-adjustable skirt 3.

Referring back to FIG. 1, the preferred embodiment of the first skirt-positioning mechanism 4 may utilize manual or automatic actuation mechanisms. A possible manually actuating embodiment of the first skirt-positioning mechanism 4 comprises a first linkage arm 41, wherein the first length-adjustable skirt 2 is terminally and rotatably connected to the first linkage arm 41. Further, the platform 1 is laterally connected to the first linkage arm 41, offset from the first length-adjustable skirt 2. The first linkage arm 41 allows the first angle 43 to be increased or decreased at discrete angular values. For example, first linkage arm 41 may increase or decrease the first angle 43 by increments of five degrees. Once, the first angle 43 is chosen, the first linkage arm 41 locks the angular position of the first length-adjustable skirt 2 in relation to the platform 1.

As can be seen in FIG. 2, in order to accommodate animals of various sizes, the first length-adjustable skirt 2 and the second length-adjustable skirt 3 are rotatably mounted onto the platform 1. More specifically, the first length-adjustable skirt 2 is hingedly mounted onto the platform 1 about a first rotation axis 21, while the second length-adjustable skirt 3 is hingedly mounted onto the platform 1 about a second rotation axis 31. To preserve the alignment between the first length-adjustable skirt 2 and the second length-adjustable skirt 3, the first rotation axis 21 and the second rotation axis 31 are positioned parallel and offset from each other. As a result, reducing the first angle 43 and the second angle 53 increases the effective height and volume of the holding area. Likewise, increasing the first offset distance 42 and the second offset distance 52 increases the effective width of the holding area, and thus the effective volume. This creates a deep and wide holding area which may be used to restrain a large animal. In contrast, increasing the first angle 43 and the second angle 53 reduces the effective height and volume of holding area. Likewise, reducing the first offset distance 42 and the second offset distance 52 reduces the effective width of the holding area, and thus the effective volume. This may be used to create a short, narrow holding area which may be used to restrain a thin animal with short limbs.

As can be seen in FIG. 2, the first skirt-positioning mechanism 4 further comprises a plurality of first linear-offset holes 44, a plurality of first angular-offset holes 45, and a first fastener 46. The first fastener 46 allows one of the plurality of first linear-offset holes 44 to interlock with the one of the plurality of first angular-offset holes 45, thereby setting the first angle 43 between the first length-adjustable skirt 2 and the platform 1. The preferred first fastener 46 is a knurled ring knob with a gripping surface for ease of insertion into the plurality of first angular-offset holes 45. Accordingly, the plurality of first angular-offset holes 45 laterally traverses through the first linkage arm 41. This allows the first fastener 46 to traverse through the first linkage arm 41 and connect to the plurality of first linear-offset holes 44. Additionally, the plurality of first angular-offset holes 45 is distributed along the first linkage arm 41. This allows the first angle 43 to be affixed at discrete angular values. The plurality of first linear-offset holes 44 laterally traverses into the platform 1, adjacent to the first rotation axis 21. This allows a selected hole from the plurality of first linear-offset holes 44 to be positioned concentric to a selected hole from the plurality of first angular-offset holes 45. As a result, the first fastener 46 can be releasably engaged to a selected hole from the plurality of first linear-offset holes 44 and a selected hole from the plurality of first angular-offset holes 45.

The second skirt-positioning mechanism 5 comprises a second linkage arm 51, wherein the second length-adjustable skirt 3 is terminally and rotatably connected to the second linkage arm 51. Further, the platform 1 is laterally connected to the second linkage arm 51, offset from the second length-adjustable skirt 3. Similar to the first linkage arm 41, this allows the second linkage arm 51 to engage the platform 1. The second linkage arm 51 increments the second angle 53 by discrete angular values.

Referring back to FIG. 2, the second skirt-positioning mechanism 5 further comprises a plurality of second linear-offset holes 54, a plurality of second angular-offset holes 55, and a second fastener 56. The plurality of second angular-offset holes 55 laterally traverses through the second linkage arm 51, thereby allowing the second fastener 56 to traverse through the second linkage arm 51 and engage the plurality of second linear-offset holes 54. Additionally, the plurality of second angular-offset holes 55 is distributed along the second linkage arm 51. Each angular-offset hole corresponds to a discrete angular value for the second angle 53. The plurality of second linear-offset holes 54 laterally traverses into the platform 1, adjacent to the second rotation axis 31. As such, the second fastener 56 inserts into the plurality of second linear-offset holes 54 through the plurality of second angular-offset holes 55. The second fastener 56 is releasably engaged to a selected hole from the plurality of second linear-offset holes 54 and a selected hole from the plurality of second angular-offset holes 55. As a result, an interlocking engagement is formed between a selected hole from the plurality of second linear-offset holes 54 and a selected hole from the plurality of second angular-offset holes 55 and the platform 1, which affixes the second angle 53 between the second length-adjustable skirt 3 and the platform 1.

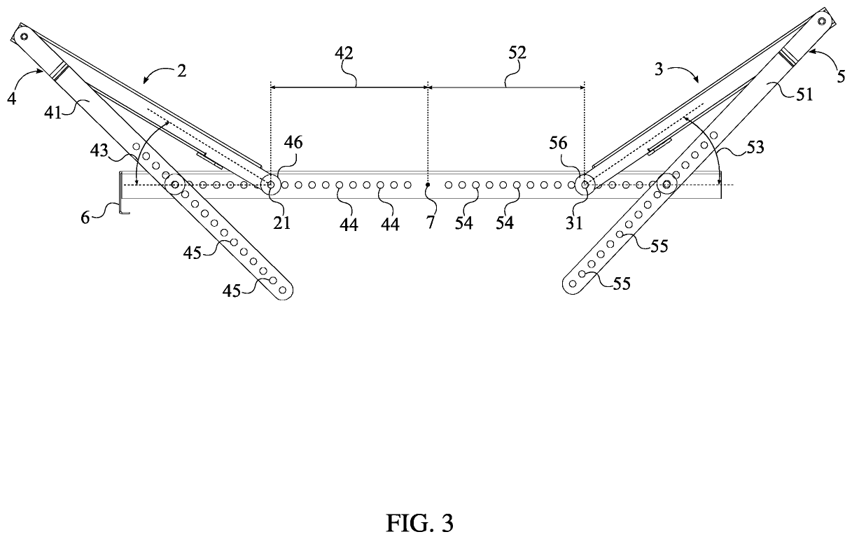

As can be seen in FIG. 3, the first angle 43 can be adjusted by switching the one of the plurality of first angular-offset holes 45 that is engaged to one of the plurality of first linear-offset holes 44. For example, selecting a first angular-offset hole located closer to the first length-adjustable skirt 2 lowers the first angle 43. Similarly, the second angle 53 can be adjusted by switching one of the plurality of second angular-offset holes 55 that is engaged to one of the plurality of second linear-offset holes 54. More specifically, selecting a second angular-offset hole located closer to the second length-adjustable skirt 3 lowers the second angle 53. Lowering the first angle 43 and the second angle 53 increases the effective volume enclosed by the holding area. In contrast, increasing the first angle 43 and the second angle 53 reduces the effective volume enclosed by the holding area. This is achieved by selecting a first angular-offset hole located farther from the first length-adjustable skirt 2, which increases the first angle 43. Similarly, selecting a second angular-offset hole located farther from the second length-adjustable skirt 3 increases the second angle 53. Together, this can be used to increase the effective volume enclosed by the holding area.

The volume of the holding area can be changed by adjusting the first offset distance 42 and the second offset distance 52. This is achieved by selecting a first linear-offset hole located closer to the central axis 7, which reduces the first offset distance 42. Similarly, selecting a second linear-offset hole located closer to the central axis 7 reduces the second offset distance 42. This brings the first length-adjustable skirt 2 and the second length-adjustable skirt 3 closer together, which reduces the volume of the holding area enclosed between. In contrast, selecting a first linear-offset hole located farther from to the central axis 7 increases the first offset distance 42. Similarly, selecting a second linear-offset hole located farther to the central axis 7 increases the second offset distance 42. This moves the first length-adjustable skirt 2 and the second length-adjustable skirt 3 father apart, which increases the volume of the holding area enclosed between.

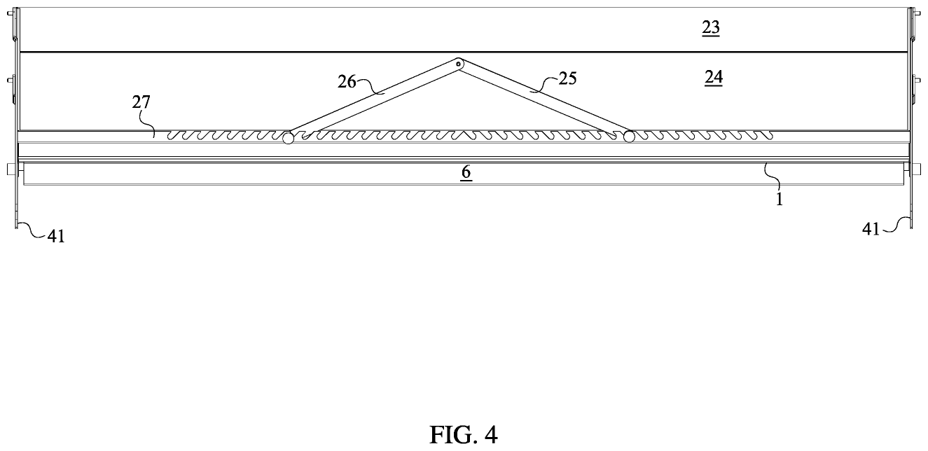

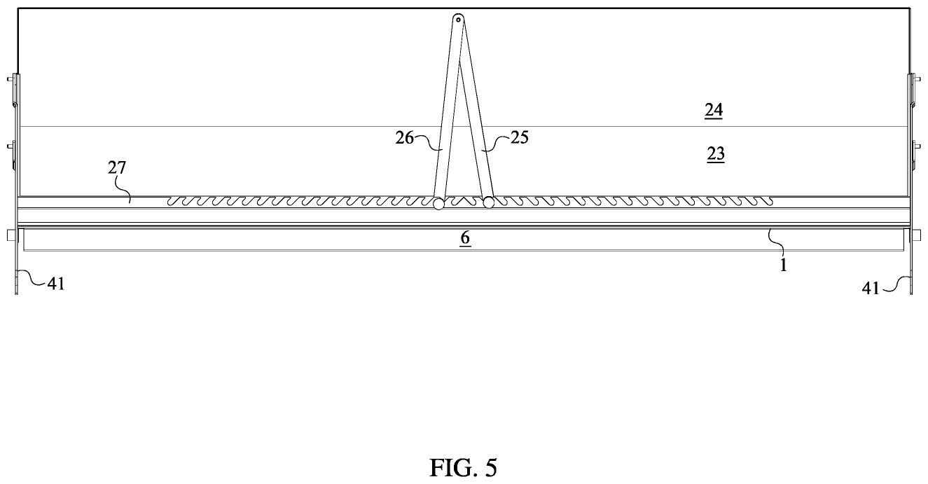

As can be seen in FIG. 4-FIG. 5, an embodiment of the first length-adjustable skirt 2 and the second length-adjustable skirt 3 allows them to be manually extended thereby increasing their effective length. As such, the first length-adjustable skirt 2 and the second length-adjustable skirt 3 each comprise a skirt base 23, a skirt extension 24, a front pawl arm 25, a rear pawl arm 26, and a set of ratcheting teeth 27. The skirt extension 24 is slidably mounted to the skirt base 23, wherein the skirt extension 24 extends out of the skirt base 23 to increase the effective length of the first length-adjustable skirt 2 or the second length-adjustable skirt 3. The set of ratcheting teeth 27 is integrated along the skirt base 23. The set of ratcheting teeth 27 locks the position of the front pawl arm 25 and the rear pawl arm 26. As such, a distal end of the front pawl arm 25 and a distal end of the rear pawl arm 26 are hingedly connected to each other. This allows the front pawl arm 25 to rotate in relation to the rear pawl arm 26 which causes the angle between the front pawl arm 25 and the rear pawl arm 26 to change. Further, the distal end of the front pawl arm 25 and the distal end of the rear pawl arm 26 are pressed against the skirt extension 24. This allows the front pawl arm 25 and the rear pawl arm 26 to lift the skirt extension 24 out of the skirt base 23.

Referring more specifically to FIG. 5, a proximal end of the front pawl arm 25 is releasably engaged to a selected tooth from the set of ratcheting teeth 27, while a proximal end of the rear pawl arm 26 is releasably engaged to a selected tooth from the set of ratcheting teeth 27. This allows the extension length of the skirt extension 24 to be incremented according to the distance between the selected teeth. The front pawl arm 25 and the rear pawl arm 26 can be positioned in a substantially vertical orientation by mounting the proximal ends of the front pawl arm 25 and the rear pawl arm 26 directly adjacent to each other. This maximizes the distance between the pivot axis of the front pawl arm 25 and the rear pawl arm 26 and the set of ratcheting teeth 27, thereby causing the skirt extension 24 to extend out of the skirt base 23. Referring more specifically to FIG. 4, mounting the proximal ends of the front pawl arm 25 and the rear pawl arm 26 at opposite ends of the set of ratcheting teeth 27 positions the front pawl arm 25 and the rear pawl arm 26 at a slanted orientation. This shortens the distance between the set of ratcheting teeth 27 and the pivot axis, thereby retracting the skirt extension 24 into the skirt base 23.

In reference to FIG. 6, possible automatically-actuating embodiments of the first skirt-positioning mechanism 4 and the second skirt-positioning mechanism 5 may utilize electromechanical or hydraulic mechanisms. The first skirt-positioning mechanism 4 comprises a first linear actuator 47 and a first angular-adjustment motor 48. The first linear actuator 47 adjusts the first offset-distance 42 of the first length-adjustable skirt 2 while the first angular-adjustment motor 48 adjusts the first angle 43 between the first length-adjustable skirt 2 and the platform 1. Accordingly, a fixed end of the first linear actuator 47 is mounted adjacent to the surgical-table adapter 6, offset from the platform 1. The first length-adjustable skirt 2 is operatively coupled to a driving end of the first linear actuator 47, wherein the first linear actuator 47 is used to move the first length-adjustable skirt 2 and the first angular-adjustment motor 48 along the platform 1.

Additionally, the first angular-adjustment motor 48 is operatively integrated between the first length-adjustable skirt 2 and the driving end of the first linear actuator 47, wherein the first angular-adjustment motor 48 is used to rotate the first length-adjustable skirt 2 about the first rotation axis 21. Furthermore, the rotational output of the first angular-adjustment motor 48 is integrated to the first length-adjustable skirt 2, wherein the rotational output is used to adjust the first angle 43. The angular-adjustment motor can also lock the first angle 43 between the first length-adjustable skirt 2 and the platform 1. This is achieved through the use of acme threads, which prevents the rotational output from moving when the first angular-adjustment motor 48 is not powered.

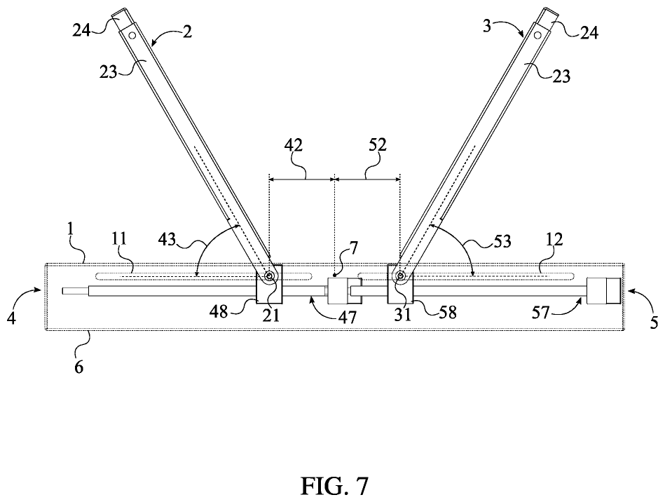

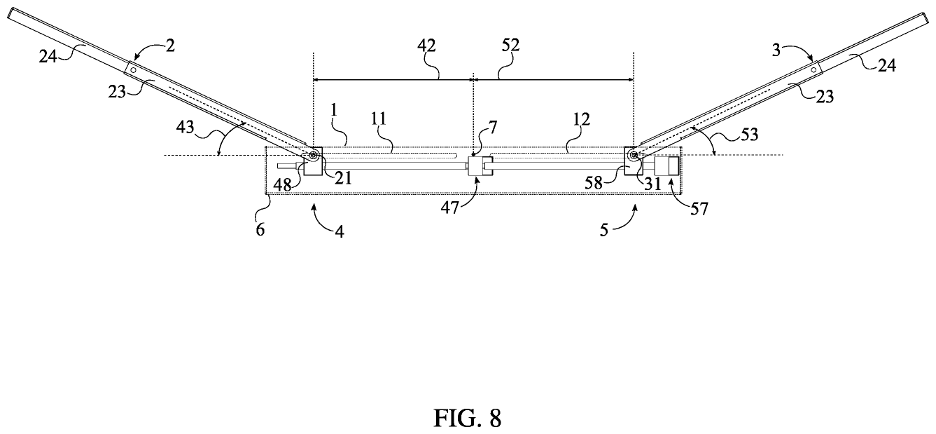

Referring to FIG. 7-8, the first skirt-positioning mechanism 4 further comprises a first elongated slot 11. The first elongated slot 11 is cut through platform 1 to allow the rotational output of the motor to traverse therethrough. This allows the first angular-adjustment motor 48 to rotatably connect to the first length-adjustable skirt 2 through the first elongated slot 11.

The second skirt-positioning mechanism 5 comprises a second linear actuator 57 and a second angular-adjustment motor 58. Like the first skirt-positioning mechanism 4, the second linear actuator 57 and the second angular-adjustment motor 58 control the second angle 53 and the second offset distance 52 between the second length-adjustable skirt 3 and the platform 1. Additionally, a fixed end of the second linear actuator 57 is mounted adjacent to the surgical-table adapter 6, offset from the platform 1. This positions the second linear actuator 57 opposite to the first linear actuator 47, capable of shifting the second length-adjustable skirt 3 away from the first length-adjustable skirt 2. The second length-adjustable skirt 3 is operatively coupled to a driving end of the second linear actuator 57, wherein the second linear actuator 57 is used to move the second length-adjustable skirt 3 along the platform 1. This allows the second linear actuator 57 to adjust the second offset distance 52. The second angular-adjustment motor 58 is operatively integrated between the second length-adjustable skirt 3 and the driving end of the second linear actuator 57, wherein the second angular-adjustment motor 58 is used to rotate the second length-adjustable skirt 3 about the second rotation axis 31. In particular, the second angular-adjustment motor 58 controls the second angle 53 between the second length-adjustable skirt 3 and the platform 1. Further, the second angular-adjustment motor 58 can affix the second angle 53, thereby fixing the dimensions of the holding area enclosed by the first length-adjustable skirt 2 and the second length-adjustable skirt 3.

The second skirt-positioning mechanism 5 further comprises a second elongated slot 12. The second elongated slot 12 is cut through platform 1 to allow the rotational output of the motor to traverse therethrough. This allows the second angular-adjustment motor 58 to rotatably connect to the second length-adjustable skirt 3 through the second elongated slot 12.

The preferred implementation of the first linear actuator 47 and the second linear actuator 57 each comprise a shaft rotatably coupled to a motor. The shaft allows the first angular-adjustment motor 48 and the second angular-adjustment motor 58 to operatively couple to the motor. More specifically, the first angular-adjustment motor 48 is mounted onto the shaft of the first linear actuator 47. Similarly, the second angular-adjustment motor 58 is mounted onto the shaft of the second linear actuator 57. As the motor rotates the shaft, the first angular-adjustment motor 48 and the second angular-adjustment motor 58 shifts along the length of the shaft of the first linear actuator 47 and the second linear actuator 57.

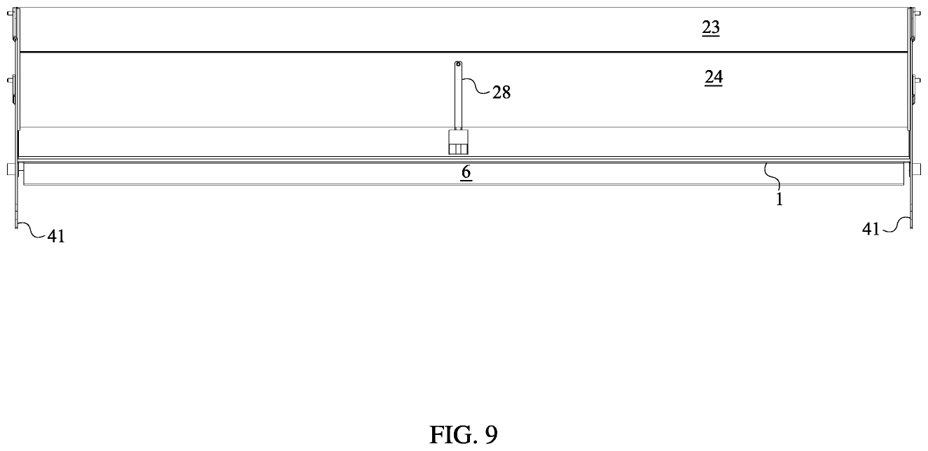

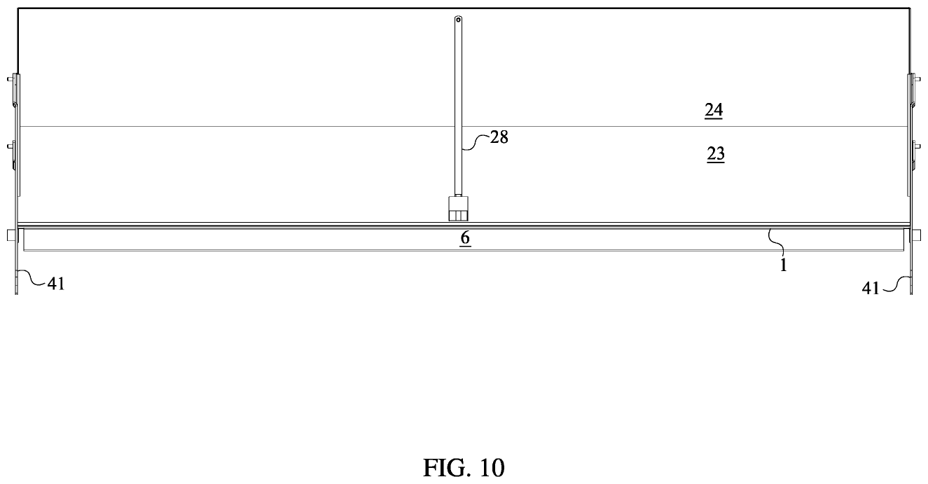

In reference to FIG. 9-10, another possible embodiment of the first length-adjustable skirt 2 and the second length-adjustable skirt 3 each comprise a skirt base 23, a skirt extension 24, and a motorized lifting mechanism 28. The skirt extension 24 is slidably mounted to the skirt base 23. This enables a motorized mechanism for extending the skirt extension 24 out of the skirt base 23 increases the effective volume enclosed by the holding area. More specifically, the motorized lifting mechanism 28 is operatively integrated between the skirt extension 24 and the skirt base 23, wherein the motorized lifting mechanism 28 is used to offset the skirt extension 24 from the skirt base 23. Alternately, a manual lifting mechanism may also be used in combination with the motorized embodiment of the first skirt-positioning mechanism 4 and the second skirt-positioning mechanism 5.

Although the invention has been explained in relation to its preferred embodiment, it is to be understood that many other possible modifications and variations can be made without departing from the spirit and scope of the invention as hereinafter claimed.

What is claimed is:

1. A side-attachment cradle for veterinary surgical tables comprises:
a platform;
a first length-adjustable skirt;
a second length-adjustable skirt;
at least one first skirt-positioning mechanism;
at least one second skirt-positioning mechanism;
a surgical-table adapter;
the first length-adjustable skirt and the second length-adjustable skirt being positioned adjacent to the platform;
the first length-adjustable skirt being hingedly mounted onto the platform about a first rotation axis;
the second length-adjustable skirt being hingedly mounted onto the platform about a second rotation axis;
the first rotation axis and the second rotation axis being positioned parallel and offset from each other;
the first skirt-positioning mechanism being operatively integrated between the first length-adjustable skirt and the platform;
the second skirt-positioning mechanism being operatively integrated between the second length-adjustable skirt and the platform; and
the surgical-table adapter being mounted adjacent to the platform, opposite the first length-adjustable skirt and the second length-adjustable skirt.

2. The side-attachment cradle for veterinary surgical tables as claimed in claim 1, wherein the first skirt-positioning mechanism is used to adjust a first angle between the first length-adjustable skirt and the platform, and wherein the first skirt-positioned mechanism is used to adjust a first offset distance between the first rotation axis and a central axis of the platform.

3. The side-attachment cradle for veterinary surgical tables as claimed in claim 1, wherein the second skirt-positioning mechanism is used to adjust a second angle between the second length-adjustable skirt and the platform, and wherein the second skirt-positioned mechanism is used to adjust a second offset distance between the second rotation axis and the central axis of the platform.

4. The side-attachment cradle for veterinary surgical tables as claimed in claim 1 comprises:
the at least one first skirt-positioning mechanism being a pair of first skirt-positioning mechanisms; and
the pair of first skirt-positioning mechanisms being positioned opposite each other across the platform.

5. The side-attachment cradle for veterinary surgical tables as claimed in claim 1 comprises:
the at least one first skirt-positioning mechanism being a pair of first skirt-positioning mechanisms; and
the pair of first skirt-positioning mechanisms being positioned opposite each other across the platform.

6. The side-attachment cradle for veterinary surgical tables as claimed in claim 1 comprises:
the first skirt-positioning mechanism comprises a first linkage arm;
the first length-adjustable skirt being terminally and rotatably connected to the first linkage arm; and
the platform being laterally connected to the first linkage arm, offset from the first length-adjustable skirt.

7. The side-attachment cradle for veterinary surgical tables as claimed in claim 6 comprises:
the first skirt-positioning mechanism further comprises a plurality of first linear-offset holes, a plurality of first angular-offset holes, and a first fastener;
the plurality of first angular-offset holes laterally traversing through the first linkage arm;
the plurality first angular-offset holes being distributed along the first linkage arm;
the plurality of first linear-offset holes laterally traversing into the platform, adjacent to the first rotation axis; and
the first fastener being releasably engaged to a selected hole from the plurality of first linear-offset holes and a selected hole from the plurality of angular-offset holes.

8. The side-attachment cradle for veterinary surgical tables as claimed in claim 1 comprises:
the second skirt-positioning mechanism comprises a second linkage arm;
the second length-adjustable skirt being terminally and rotatably connected to the second linkage arm; and
the platform being laterally connected to the second linkage arm, offset from the second length-adjustable skirt.

9. The side-attachment cradle for veterinary surgical tables as claimed in claim 8 comprises:
the second skirt-positioning mechanism further comprises a plurality of second linear-offset holes, a plurality of second angular-offset holes, and a second fastener;
the plurality of second angular-offset holes laterally traversing through the second linkage arm;
the plurality second angular-offset holes being distributed along the second linkage arm;
the plurality of second linear-offset holes laterally traversing into the platform, adjacent to the second rotation axis; and
the second fastener being releasably engaged to a selected hole from the plurality of second linear-offset holes and a selected hole from the plurality of second angular-offset holes.

10. The side-attachment cradle for veterinary surgical tables as claimed in claim 1 comprises:
the first skirt-positioning mechanism comprises a first linear actuator and a first angular-adjustment motor;
a fixed end of the first linear actuator being mounted adjacent to the surgical-table adapter, offset from the platform;
the first length-adjustable skirt being operatively coupled to a driving end of the first linear actuator, wherein the first linear actuator is used to move the first length-adjustable skirt along the platform; and
the first angular-adjustment motor being operatively integrated between the first length-adjustable skirt and the driving end of the first linear actuator, wherein the first angular-adjustment motor is used to rotate the first length-adjustable skirt about the first rotation axis.

11. The side-attachment cradle for veterinary surgical tables as claimed in claim 10 comprises:
the first skirt-positioning mechanism further comprises a first elongated slot; and
the first angular-adjustment motor being rotatably connected to the first length-adjustable skirt through the first elongated slot.

12. The side-attachment cradle for veterinary surgical tables as claimed in claim 1 comprises:
the second skirt-positioning mechanism comprises a second linear actuator and a second angular-adjustment motor;
a fixed end of the second linear actuator being mounted adjacent to the surgical-table adapter, offset from the platform;
the second length-adjustable skirt being operatively coupled to a driving end of the second linear actuator, wherein the second linear actuator is used to move the second length-adjustable skirt along the platform; and
the second angular-adjustment motor being operatively integrated between the second length-adjustable skirt and the driving end of the second linear actuator, wherein the second angular-adjustment motor is used to rotate the second length-adjustable skirt about the second rotation axis.

13. The side-attachment cradle for veterinary surgical tables as claimed in claim 12 comprises:
the second skirt-positioning mechanism further comprises a second elongated slot; and
the second angular-adjustment motor being rotatably connected to the second length-adjustable skirt through the second elongated slot.

14. The side-attachment cradle for veterinary surgical tables as claimed in claim 1 comprises:
the first length-adjustable skirt and the second length-adjustable skirt each comprise a skirt base, a skirt extension, a front pawl arm, a rear pawl arm, and a set of ratcheting teeth;
the skirt extension being slidably mounted to the skirt base;
the set of ratcheting teeth being integrated along the skirt base;
a distal end of the front pawl arm and a distal end of the rear pawl arm being hingedly connected to each other;
the distal end of the front pawl arm and the distal end of the rear pawl arm being pressed against the first skirt extension;
a proximal end of the front pawl arm being releasably engaged to a selected tooth from the set of ratcheting teeth; and
a proximal end of the rear pawl arm being releasably engaged to a selected tooth from the set of ratcheting teeth.

15. The side-attachment cradle for veterinary surgical tables as claimed in claim 1 comprises:
the first length-adjustable skirt and the second length-adjustable skirt each comprise a skirt base, a skirt extension, and a motorized lifting mechanism;
the skirt extension being slidably mounted to the skirt base; and
the motorized lifting mechanism being operatively integrated between the skirt extension and the skirt base, wherein the motorized lifting mechanism is used to offset the skirt extension from the skirt base.
